(19) United States
(12) Patent Application Publication
(10) Pub. No.: US 2023/0368645 A1
(43) Pub. Date: Nov. 16, 2023

(54) HEADWALL BEACON SYSTEM

(71) Applicant: Stryker Corporation, Kalamazoo, MI (US)

(72) Inventors: Krishna Sandeep Bhimavarapu, Kalamazoo, MI (US); Annie Désaulniers, Bothell, WA (US); Jerald A. Trepanier, Augusta, MI (US); Scott Austin, Kalamazoo, MI (US)

(21) Appl. No.: 18/028,586

(22) PCT Filed: Oct. 7, 2021
(86) PCT No.: PCT/US2021/053878

§ 371 Date: Mar. 27, 2023

Related U.S. Application Data
(60) Provisional application No. 63/091,019, filed on Oct. 13, 2020.

(51) Int. Cl. G08B 26/00; G16H 40/67; G16H 40/20

(52) U.S. Cl. CPC G08B 26/008; G16H 40/67; G16H 40/20

(57) ABSTRACT

A stationary communication unit is adapted to be mounted to a headwall of a healthcare facility and forward messages between a patient support apparatus and a nurse call system. The unit includes first and second transceivers. The first transceiver may use two different communication protocols and forward audio signals from a nurse call system to the patient support apparatus (and vice versa) using a first one of the communication protocols, and emit a beacon signal using the second communication protocol. The beacon signal is adapted to be detected by electronic devices positioned in the room. The second transceiver sends a first identifier to the patient support apparatus and the first transceiver sends a second identifier to any electronic devices positioned in the room. The communication unit may also forward sensor readings to the patient support apparatus and/or to a hospital network, either directly or via a mesh network.

### CROSS-REFERENCE TO RELATED APPLICATIONS

This application claims priority to U.S. provisional patent application Ser. No. 63/091,019 filed Oct. 13, 2020, by inventors Krishna Bhimavarapu et al. and entitled HEADWALL BEACON SYSTEM, the complete disclosure of which is incorporated herein by reference.

### BACKGROUND

The present disclosure relates to medical facilities having headwalls with one or more communication outlets that enable communication between a patient support apparatus (e.g. a bed, stretcher, cot, recliner, wheelchair, etc.) and one or more devices that are coupled to the communication outlet (e.g. a nurse call system, entertainment controls, room controls, etc.).

Medical facilities, such as hospitals, typically include a headwall having one or more communication outlets into which the plugs of cables connected to beds and/or pendants can be inserted. For example, hospital headwalls will typically include at least one outlet that interfaces with a nurse call system and which is designed to accept a cable from a hospital bed, or from a handheld pendant positioned on the bed. When the cable is plugged into this outlet, a patient positioned on the bed is able to press a button to summon a nurse and/or to communicate aurally with the nurse.

Existing headwall outlets also typically communicate with one or more environmental controls, such as one or more controls for in-room televisions and/or room lights. When the appropriate device and its associated cable are plugged into the headwall connector from a bed, pendant, or other device, a person is able to control the environmental control via the device (e.g. bed, pendant, or other device). Thus, for example, a patient positioned on a bed is able to control the volume of a television in the room via controls on the bed due to a cable being connected from the bed to the headwall outlet. In some instances, a single cable is plugged into a single outlet on the headwall and used for communicating both with the nurse call system of the medical facility, and for communicating with the one or more environmental controls. In such instances, the headwall outlet is coupled to an appropriate circuit included within the infrastructure of the healthcare facility that forwards the environmental control signals to the appropriate environmental control unit, and forwards the nurse call signals to the appropriate component of the nurse call system.

### SUMMARY

A stationary communication unit adapted to be mounted in a room of a healthcare facility is provided that enables wireless communication between the patient support apparatus and a nurse call outlet built into the headwall of the room. The nurse call outlet may be a conventional outlet that is communicatively coupled to a conventional nurse call system, a room entertainment device (e.g. a television), and/or a room environmental control (e.g. a thermostat). In addition to enabling communication between the patient support apparatus and these devices, the stationary communication unit is adapted to emit a beacon signal that may be utilized by one or more non-patient support apparatus devices. These non-patient support apparatus devices may utilize the beacon signal for determining their own location. In some situations, these non-patient support apparatus devices may include one or more medical devices. In other situations, these non-patient support apparatus devices may include portable electronic devices carried by individuals, including, but not limited to, visitors, caregivers, and/or other healthcare personnel. In some embodiments, the portable electronic devices may include smart phones, computer tablets and/or other devices that utilize one or more software apps that communicate with the beacon signals for providing location information to the user, including, but not limited to, instructions for traveling to a destination in the healthcare facility. In some embodiments, the stationary communication unit may include one or more sensors for sensing environmental conditions (e.g. noise, temperature, smell, etc.) and reporting this location to healthcare personnel for evaluation of the comfort levels provided to patients.

According to one embodiment of the present disclosure, a stationary communication unit adapted to be mounted in a room of a healthcare facility is provided. The stationary communication unit includes a first transceiver, a second transceiver, a third transceiver, and a controller. The first transceiver is adapted to wirelessly communicate over a first communication channel with a patient support apparatus positioned in the room. The second transceiver is adapted to wirelessly communicate with the patient support apparatus over a second communication channel different from the first communication channel. The second transceiver is further adapted to transmit a message to the patient support apparatus over the second communication channel that includes a first identifier associated with the stationary communication unit. The third transceiver is in communication with a nurse call system outlet that is installed on a wall of the room and that is communicatively coupled to a nurse call system. The third transceiver is adapted to receive audio signals from a wire of the nurse call system outlet. The controller is adapted to forward the audio signals received from the third transceiver to the patient support apparatus over the first communication channel. The controller is further adapted to periodically emit a beacon signal from the first transceiver wherein the beacon signal includes a second identifier adapted to be detected by an electronic device other than the patient support apparatus positioned in the room.

According to other aspects of the present disclosure, the first identifier and the second identifier may be the same, or they may be different.

In some embodiments, the second identifier is a room number for the room.

The first transceiver, in some embodiments, is a Bluetooth transceiver and the second transceiver is an infrared transceiver. In such embodiments, the first transceiver may use a Bluetooth classic protocol for the first communication channel and a Bluetooth Low Energy protocol for transmitting the second identifier.

In some embodiments, the communication unit further includes a sensor in communication with the controller. The sensor is adapted to detect a parameter relating to the room and the controller is adapted to forward readings from the sensor to an off-board device using at least one of the first transceiver or the second transceiver. In some such embodiments, the sensor is a sound sensor adapted to detect sound in the room and the off-board device is the patient support apparatus. Alternatively, or additionally, the sensor may be a light sensor adapted to detect an amount of light in the room and the off-board device is the patient support apparatus.

In some embodiments, the controller is further adapted to include the first identifier in the audio signals forwarded to the patient support apparatus via the first transceiver.

The electronic device, in some embodiments, includes a smart phone.

The off-board device is a second stationary communication unit different from the stationary communication unit, in some embodiments.

According to another aspect of the present disclosure, a stationary communication unit adapted to be mounted in a room of a healthcare facility is provided. The stationary communication unit includes a first transceiver, a second transceiver, a third transceiver, a sensor, and a controller. The first transceiver is adapted to wirelessly communicate over a first communication channel with a patient support apparatus positioned in the room. The second transceiver is adapted to wirelessly communicate with the patient support apparatus over a second communication channel different from the first communication channel. The second transceiver is further adapted to transmit a message to the patient support apparatus over the second communication channel that includes a first identifier associated with the stationary communication unit. The third transceiver is in communication with a nurse call system outlet that is installed on a wall of the room and that is communicatively coupled to a nurse call system. The third transceiver is adapted to receive audio signals from a wire of the nurse call system outlet. The sensor is adapted to detect a parameter relating to the room. The controller is adapted to forward the audio signals received from the third transceiver to the patient support apparatus over the first communication channel, as well as to forward readings from the sensor to an off-board device.

In some embodiments, the first transceiver is a Bluetooth transceiver, the second transceiver is an infrared transceiver, and the controller is adapted to forward the readings from the sensor to the off-board device using the Bluetooth transceiver.

The off-board device may be the patient support apparatus.

In some embodiments, the sensor is a sound sensor adapted to detect sound in the room and the off-board device is the patient support apparatus. Alternatively, or additionally, the sensor may be a light sensor adapted to detect an amount of light in the room and the off-board device is the patient support apparatus.

The stationary communication unit, in some embodiments, further comprises a WiFi transceiver adapted to communicate with a wireless access point of a local area network. In such embodiments, the off-board device is a server communicatively coupled to the local area network.

In some embodiments, the first transceiver is adapted to transmit signals using a first protocol and a second protocol different from the first protocol, and the controller is adapted to forward the audio signals received from the third transceiver to patient support apparatus using the first transceiver and the first protocol. The controller may further be adapted to periodically emit a beacon signal from the first transceiver using the second protocol, wherein the beacon signal includes a second identifier adapted to be detected by electronic devices positioned in the room.

In some embodiments, the first identifier and the second identifier are the same, while in other embodiments, the two identifiers are different.

The off-board device is a second stationary communication unit different from the stationary communication unit, in some embodiments.

According to yet another aspect of the present disclosure, a first stationary communication unit adapted to be mounted in a room of a healthcare facility is provided. The first stationary communication unit includes a first transceiver, a second transceiver, a third transceiver, and a controller. The first transceiver is adapted to wirelessly communicate over a first communication channel with a patient support apparatus positioned in the room. The second transceiver is adapted to wirelessly communicate with the patient support apparatus over a second communication channel different from the first communication channel. The second transceiver is further adapted to transmit a message to the patient support apparatus over the second communication channel that includes a first identifier associated with the first stationary communication unit. The third transceiver is in communication with a nurse call system outlet that is installed on a wall of the room and that is communicatively coupled to a nurse call system. The third transceiver is adapted to receive audio signals from a wire of the nurse call system outlet. The controller is adapted to forward the audio signals received from the third transceiver to the patient support apparatus over the first communication channel, as well to transmit data to a second stationary communication unit using the first transceiver. The second stationary communication unit is different from the first stationary communication unit.

According to still other aspects of the present disclosure, the first stationary communication unit may include a sensor in communication with the controller wherein the sensor is adapted to detect a parameter relating to the room, and wherein the data transmitted to the second stationary communication unit includes data from the sensor. The sensor may be a sound sensor, a temperature sensor, and/or a light sensor.

In some embodiments, the controller is further adapted to only transmit the data from the sensor to the secondary communication unit if the sensor detects a parameter that exceeds a threshold, thereby avoiding transmissions of data when the parameter is below the threshold.

In some embodiments, the first stationary communication unit further includes a clock, and the controller is further adapted to transmit a time signal from the clock along with the data from the sensor that is transmitted to the secondary communication unit.

In some embodiments, the controller is further adapted to include a second identifier with the data transmitted to the second stationary communication unit. The second identifier indicates the room in which the first stationary communication unit is located.

In some embodiments, the controller is further adapted to receive sensor data from the second stationary communication unit via the first transceiver, and to forward the sensor data to a third stationary communication unit via the first transceiver.

In some embodiments, the controller is further adapted to periodically emit a beacon signal from the first transceiver, wherein the beacon signal includes a second identifier adapted to be detected by electronic devices positioned in the room.

In some embodiments, the first transceiver is a Bluetooth transceiver and the second transceiver is an infrared transceiver.

The first transceiver, in some embodiments, is adapted to receive audio signals from the patient support apparatus and forward the received audio signals to the nurse call system outlet using the third transceiver, thereby enabling two-way audio communication between the patient support apparatus and the nurse call system outlet.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration is used in the description herein of various embodiments (e.g. first, second, third, etc.). Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first embodiment of the disclosure;

FIG. 2 is a perspective view of the patient support apparatus of FIG. 1 shown in a hospital room adjacent a headwall unit comprised of separate first and second wall units;

FIG. 3 is perspective view of an alternative headwall unit that may be used in place of the first and second wall units of FIG. 2;

FIG. 4 is a block diagram one embodiment of the headwall unit of FIG. 3 shown communicatively coupled to an illustrative IT infrastructure of a representative healthcare facility;

FIG. 5 is a block diagram of multiple ones of the headwall units of FIG. 3 shown communicatively coupled to an illustrative IT infrastructure of a representative healthcare facility; and

FIG. 6 is a perspective view of a portion of a healthcare facility showing multiple headwall units incorporated therein.

### DETAILED DESCRIPTION OF THE EMBODIMENTS

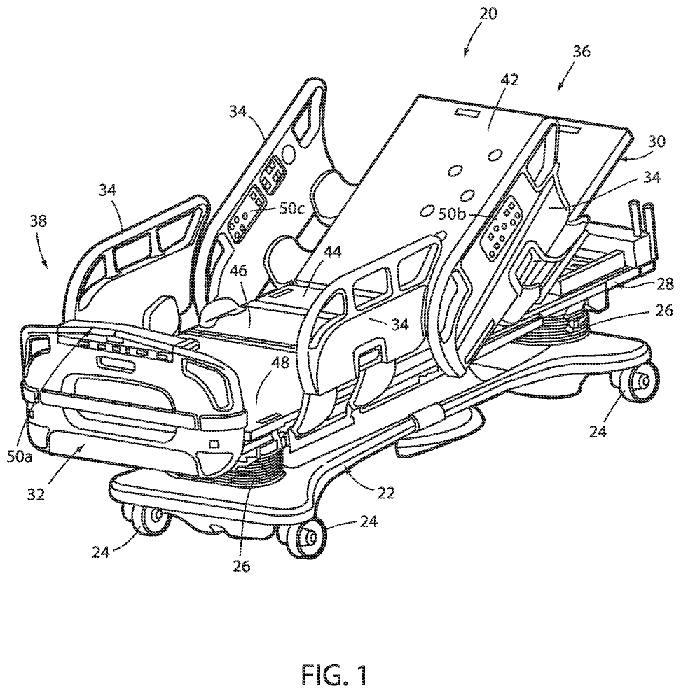

An illustrative patient support apparatus 20 according to a first embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, a wheelchair, or any other mobile structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a footboard 32 (which may be removable) and a plurality of siderails 34. Siderails 34 are all shown in a raised position in FIG. 1 but are each individually movable to a lower position in which ingress into, and egress out of, patient support apparatus 20 is not obstructed by the lowered siderails 34.

Lifts 26 are adapted to raise and lower litter frame 28 with respect to base 22. Lifts 26 may be hydraulic actuators, pneumatic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end 36 and a foot end 38, each of whose height can be independently adjusted by the nearest lift 26. Patient support apparatus 20 is designed so that when a person lies thereon, his or her head will be positioned adjacent head end 36 and his or her feet will be positioned adjacent foot end 38.

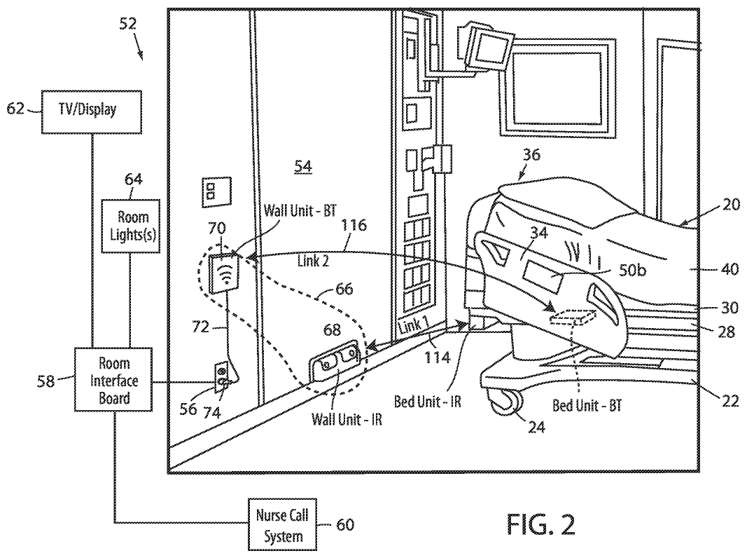

Litter frame 28 provides a structure for supporting support deck 30, footboard 32, and siderails 34. Support deck 30 provides a support surface for a mattress 40 (FIG. 2), such as, but not limited to, an air, fluid, or gel mattress. Alternatively, another type of soft cushion may be supported on support deck 30 so that a person may comfortably lie and/or sit thereon. The top surface of the mattress or other cushion forms a support surface for the patient. Support deck 30 is made of a plurality of sections, some of which are pivotable about generally horizontal pivot axes. In the embodiment shown in FIG. 1, support deck 30 includes a head section 42, a seat section 44, a thigh section 46, and a foot section 48. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

Patient support apparatus 20 further includes a plurality of control panels 50 that enable a user of patient support apparatus 20, such as a patient and/or an associated caregiver, to control one or more aspects of patient support apparatus 20. In the embodiment shown in FIG. 1, patient support apparatus 20 includes a footboard control panel 50a, a pair of outer siderail control panels 50b (only one of which is visible), and a pair of inner siderail control panels 50c (only one of which is visible). Footboard control panel 50a and outer siderail control panels 50b are intended to be used by caregivers, or other authorized personnel, while inner siderail control panels 50c are intended to be used by the patient associated with patient support apparatus 20. Not all of the control panels 50 include the same controls and/or functionality. In the illustrated embodiment, footboard control panel 50a includes a substantially complete set of controls for controlling patient support apparatus 20 while control panels 50b and 50c include a selected subset of those controls.

The controls of control panels 50 allow a user to control one or more of the following: change a height of support deck 30, raise or lower head section 42, activate and deactivate a brake for wheels 24, arm and disarm an exit detection system and, as will be explained in greater detail below, communicate with the particular IT infrastructure installed in the healthcare facility in which patient support apparatus 20 is positioned. Inner siderail control panels 50c may also include a nurse call control that enables a patient to call a nurse. A speaker and microphone are included in order to allow the patient to aurally communicate with the remotely positioned nurse.

Footboard control panel 50a is implemented in the embodiment shown in FIG. 1 as a control panel having a lid (flipped down in FIG. 1) underneath which is positioned a plurality of controls. The controls may be implemented as buttons, dials, switches, or other devices. Any of control panels 50a-c may also include a display for displaying information regarding patient support apparatus 20. The display may be a touchscreen in some embodiments.

The mechanical construction of patient support apparatus 20, as shown in FIG. 1, is the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. The construction of patient support apparatus 20 may take on a wide variety of different forms. In some embodiments, other than the components described below, patient support apparatus 20 is constructed in any of the manners described in commonly assigned, U.S. Pat. No. 8,689,376 issued Apr. 8, 2014 by inventors David Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosure of which is hereby incorporated herein by reference. In other embodiments, those components of patient support apparatus 20 not described below are constructed in any of the manners described in commonly assigned, U.S. patent application Ser. No. 13/775,285 filed Feb. 25, 2013 by inventors Guy Lemire et al. and entitled HOSPITAL BED, the complete disclosure of which is also hereby incorporated herein by reference. In still other embodiments, those components of patient support apparatus 20 not described below are constructed in any of the manners disclosed in commonly assigned, U.S. patent application Ser. No. 14/212,009 filed Mar. 14, 2014 by inventors Christopher Hough et al., and entitled MEDICAL SUPPORT APPARATUS. The mechanical construction of patient support apparatus 20 may also take on forms different from what is disclosed in the aforementioned references.

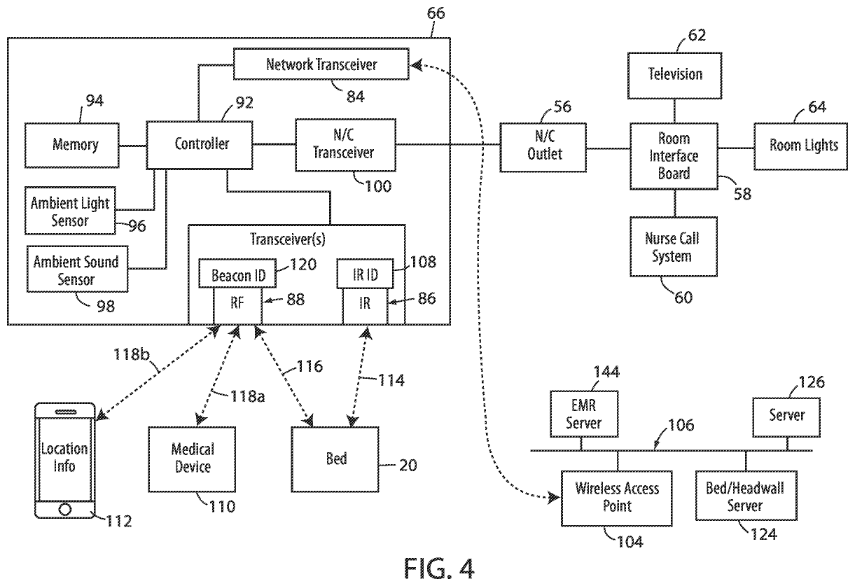

FIG. 2 illustrates patient support apparatus 20 coupled to the IT infrastructure 52 of an illustrative healthcare facility according to one common configuration. As shown therein, the healthcare facility includes a headwall 54, a nurse call outlet 56 mounted to the headwall 54, a room interface board 58 in communication with the nurse call outlet 56, and a plurality devices and components in communication with the room interface board 58, such as a nurse call system 60, a television 62, and one or more room lights 64. Nurse call outlet 56, room interface board 58, nurse call system 60, television 62, and room lights 64 may all be conventional pre-existing components that are installed in the healthcare facility independently of patient support apparatus 20 and its associated headwall units 66, as will be discussed in more detail below. Additional IT infrastructure beyond what is shown in FIG. 2 may also be present in the healthcare facility, some examples of which are discussed in more detail below with respect to FIGS. 4 & 5.

Television 62 is a conventional television, computer display, monitor, or the like that includes a display screen on which images are able to be displayed. Room lights 64 provide lighting to one or more sections of the room in which patient support apparatus 20 is located. Room lights 64 may be conventional overhead lights and/or one or more night lights or other more localized lights within the room. Nurse call system 60 may be a conventional nurse call system having one or more nurses' stations positioned throughout the healthcare facility. Nurse call system 60 routes patient calls from patient support apparatus 20 to one or more nurses' stations so that the patient is able to speak with a remotely positioned nurse at a nurses' station while the patient is supported on patient support apparatus 20, as is known in the art.

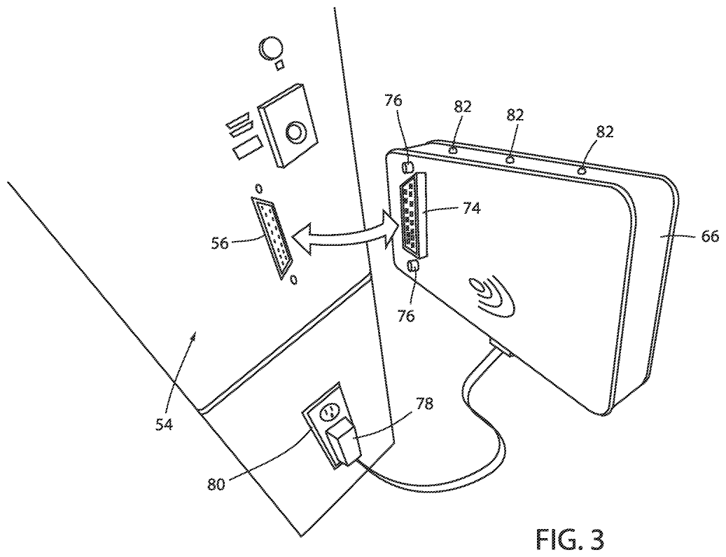

Patient support apparatus 20 is adapted to wirelessly communicate with a first wall unit 68 and a second wall unit 70. First and second wall units 68 and 70 together form a stationary headwall unit 66. In the embodiment shown in FIG. 2, headwall unit 66 is comprised of first and second wall units 68 and 70 that are physically separate from each other. In other embodiments, such as shown in FIGS. 3-6, headwall units 66 are comprised of first and second wall units 68 and 70 that are combined into a single headwall unit 66 having a single housing, as discussed in more detail below. Regardless of whether coupled together in a single housing or separated into two physically disparate units, first and second wall units 68 and 70 are adapted to communicate with each other, in at least some embodiments. Such communication takes place via a wired connection when units 68 and 70 are combined in a single housing, and may take place wirelessly when units 68 and 70 are physically separated. In still other embodiments, units 68 and 70 are not adapted to communicate with each other.

Second wall unit 70 includes a cable 72 that is coupled to nurse call outlet 56 (FIG. 2). Cable 72 allows second wall unit 70 to communicate with nurse call outlet 56 and all of the components in communication with nurse call outlet 56 (e.g. nurse call system 60, room interface board 58, etc.). Cable 72 includes a plug 74 that is adapted to mate with nurse call outlet 56. Plug 74 may vary from room to room and from healthcare facility to healthcare facility depending upon the particular type of nurse call outlet 56 that is installed within a particular room of a particular healthcare facility.

FIG. 3 illustrates an alternative embodiment of headwall unit 66 in which first and second units 68 and 70 are integrated into a single housing. Further, headwall unit 66 of FIG. 3 has been modified by the omission of cable 72. In this embodiment, the body of headwall unit 66 has plug 74 integrated therein and headwall unit 66 couples directly to nurse call outlet 56. Plug 74 is adapted to be inserted into nurse call outlet 56, which is a conventional cable interface that exists within a medical facility. Nurse call outlet 56 is a receptacle that is dimensioned and shaped to selectively and frictionally retain plug 74 therein and to support the entire second wall unit 70. One or more alignment posts 76 may be included with plug 74 in order to more securely retain headwall unit 66 to nurse call outlet 56, if desired.

In the embodiment shown in FIG. 3, plug 74 is a 37 pin connector that includes 37 pins adapted to be inserted into 37 mating sockets of nurse call outlet 56. Such 37 pin connections are one of the most common types of connectors found on existing headwalls of medical facilities for making connections to the nurse call system 60 and/or the room interface board 58. Plugs 74 of FIGS. 2 and 3 are therefore configured to mate with one of the most common type of nurse call outlets ports 56 used in medical facilities. Such 37 pin connectors, however, are not the only type of connectors, and it will be understood that headwall unit 66 can utilize different types of plugs 74 (whether integrated therein or attached to cable 72) that are adapted to electrically couple to different types of nurse call outlets 56. One example of such an alternative nurse call outlet 56 and cable is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of nurse call outlets 56 and corresponding plugs 74 may be utilized.

In the embodiment shown in FIG. 3, headwall unit 66 includes an electrical plug 78 adapted to be inserted into a conventional electrical outlet 80. Electrical plug 78 enables headwall unit 66 to receive power from the mains electrical supply via outlet 80. It will be appreciated that, in some embodiments, headwall unit 66 is battery operated and plug 78 may be omitted. In still other embodiments, headwall unit 66 may be both battery operated and include plug 78 so that, in the event of a power failure, battery power supplies power to headwall unit 66, and/or in the event of a battery failure, electrical power is received through outlet 80.

The embodiment of headwall unit 66 shown in FIG. 3 also includes a plurality of status lights 82. Status lights 82 provide visual indications about one or more aspects of headwall unit 66. For example, in some embodiments, the illumination of one of status lights 82 indicates that headwall unit 66 is in successful communication with room interface board 58 and/or patient support apparatus 20. The illumination of one or more additional status lights 82 may also or alternatively indicate that power is being supplied to headwall unit 66 and/or the status of a battery included within headwall unit 66. In addition, the illumination of another one of status lights 82 may indicate that headwall unit 66 is in successful communication with a mobile electronic device carried by a caregiver, such as, but not limited to, a smart phone, as will be discussed in greater detail below.

Headwall unit 66 (FIG. 4) is adapted to wirelessly receive signals from patient support apparatus 20 and deliver the signals to nurse call outlet 56 in a manner that matches the way the signals would otherwise be delivered to nurse call outlet 56 if a conventional nurse call cable were connected between patient support apparatus 20 and nurse call outlet 56. In other words, patient support apparatus 20 and headwall unit 66 cooperate to provide signals to nurse call outlet 56 in a manner that is transparent to nurse call outlet 56 and room interface board 58 such that these components cannot detect whether they are in communication with patient support apparatus 20 via wired or wireless communication. In this manner, a healthcare facility can utilize the wireless communication abilities of one or more patient support apparatuses 20 without having to make any changes to their existing nurse call outlets 56 (or to their nurse call system 60 or room interface boards 58).

In addition to sending signals received from patient support apparatus 20 to nurse call outlet 56, headwall unit 66 is also adapted to forward signals received from nurse call outlet 56 to patient support apparatus 20. Headwall unit 66 is therefore adapted to provide bidirectional communication between patient support apparatus 20 and nurse call outlet 56. Such bidirectional communication includes, but is not limited to, communicating audio signals between a person supported on patient support apparatus 20 and a caregiver positioned remotely from patient support apparatus 20 (which is accomplished by headwall unit 66 forwarding the audio signals of the person on patient support apparatus 20 to nurse call system 60, and vice versa).

Headwall unit 66 communicates the data and signals it receives from patient support apparatus 20 to room interface board 58 by directing the incoming data and signals it receives to the appropriate pin or pins of nurse call outlet 56. For example, when nurse call outlet 56 includes 37 sockets for coupling to a 37 pin plug, it is common for pins #30 and #31 to be used for indicating a “priority alert,” which is often synonymous with an alert that is issued when a patient exits from patient support apparatus 20. Further, depending upon the particular configuration that has been implemented at a particular healthcare facility, the connection between pins #30 and #31 may be normally open or it may be normally closed. Regardless of whether it is normally open or normally closed, whenever headwall unit 66 receives a message from patient support apparatus 20 that a person has exited from patient support apparatus 20, headwall unit 66 utilizes a nurse call transceiver 84 (discussed in more detail below' see FIG. 4) to change the status of pins #30 and #31 such that they switch from whatever state they are normally in to their opposite state. Headwall unit 66 therefore reacts to the exit message it receives from patient support apparatus 20 by either opening or closing pins #30 and #31. The nurse call system 60 that is communicatively coupled to nurse call outlet 56 interprets this opening or closing of pins #30 and #31 in the same manner as if a cable were coupled between nurse call outlet 56, such as by sending the appropriate signals to one or more nurse's stations, flashing a light outside the room of patient support apparatus 20, forwarding a call to a mobile communication device carried by the caregiver assigned to the patient of patient support apparatus 20, and/or taking other steps, depending upon the specific configuration of the nurse call system.

In addition to sending data indicating that a patient of patient support apparatus 20 has exited, or is about to exit, therefrom, patient support apparatus 20 is configured, in at least one embodiment, to wirelessly send to headwall unit 66 at least the following additional messages: messages to turn on or off one or more room lights; messages to turn on or off one or more reading lights; messages to increase or decrease the volume of a nearby television set or radio; messages to change a channel of the nearby television set or radio; and messages containing audio packets generated from one or more microphones on the patient support apparatus 20 into which the patient of patient support apparatus 20 speaks when desiring to communicate with a remote caregiver.

In other embodiments, patient support apparatus 20 is configured to wirelessly send to headwall unit 66 any one or more of the following messages, either in addition to or in lieu of any one or more of the messages just mentioned: messages indicating the current status of one or more siderails 34 of patient support apparatus 20 (e.g. whether the side rails are up or down, or have changed position); messages indicating the current status of a brake on patient support apparatus 20; messages indicating the current status of the height of support deck 30 relative to base 22 (e.g. such as whether support deck 30 is at its lowest height or not); messages indicating the current angle of head section 42; messages indicating the current status of an exit detection system (e.g. whether the exit detection system is armed or not); messages indicating the current charging status of one or more batteries on patient support apparatus 20; messages indicating the current status of an alternating current (A/C) power cable on patient support apparatus 20 (e.g. whether it is plugged in or not); diagnostic information about patient support apparatus 20; messages containing patient data gathered from one or more sensors on board patient support apparatus 20; message containing patient data gathered from one or more medical devices that are separate from patient support apparatus 20 but which communicate such data to patient support apparatus 20; and/or any other messages containing information about patient support apparatus 20, the patient supported thereon, and/or a caregiver associated with the patient.

In at least one embodiment, headwall unit 66 is further configured to transmit information to nurse call outlet 56 that does not originate from patient support apparatus 20, but instead is generated internally within headwall unit 66. For example, in one embodiment, headwall unit 66 is adapted to forward to nurse call outlet 56 a signal that indicates a “cord-out” alert whenever the communication link between headwall unit 66 and patient support apparatus 20 is unintentionally lost. In many instances, when a conventional cable is coupled between nurse call outlet 56 and a hospital bed, and the cable is inadvertently disconnected, the electrical status of pins 10 and 11 (in a conventional 37 pin connection) will be changed such that the nurse call system will recognize that the cable has become disconnected, and will therefore issue an appropriate alert to the appropriate personnel. Headwall unit 66 is configured to make the same changes to pins 10 and 11 when it unintentionally loses communication with patient support apparatus 20 that would be made to pins 10 and 11 if a cable connection between patient support apparatus 20 and nurse call outlet 56 were to become unintentionally disconnected. Thus, headwall unit 66 and patient support apparatus 20 together include the same ability to provide an indication to nurse call outlet 56 of an unintentional disconnection that exists in some currently-available cable connections to cable interfaces. Still other types of signals that originate from within headwall unit 66 may also be sent to nurse call outlet 56 in addition to, or in lieu of, this cord-out alert.

In addition to forwarding any of the above-described messages or signals to nurse call outlet 56, headwall unit 66 is also adapted, in at least one embodiment, to forward the following messages to patient support apparatus 20 based on information it receives from devices in communication with nurse call outlet 56: messages indicating the establishment and disestablishment of a nurse-call communication link (e.g. messages used for turning on and off a “nurse answer” light on patient support apparatus 20); and messages containing audio packets of a caregiver's voice (generated from a microphone into which the caregiver speaks and forwarded to the appropriate pins of nurse call outlet 56). In some embodiments, headwall unit 66 is configured to exchange audio signals between patient support apparatus 20 and nurse call outlet 56 in any of the manners disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

In other embodiments, one or more additional messages are also transmitted to patient support apparatus 20 that originate from within headwall unit 66, rather than from any of the devices in communication with nurse call outlet 56. Such messages include any one or more of the following: the charge status of a battery within headwall unit 66; acknowledgements of messages transmitted from patient support apparatus 20 to headwall unit 66; messages used to establish, maintain, and disestablish the communication link between headwall unit 66 and one or more of the following: patient support apparatus 20, a mobile electronic device associated with a caregiver, and/or one or more medical devices associated with the patient; and messages containing patient data and/or medical device data that is stored within headwall unit 66. Still other messages communicated to and/or from headwall unit 66 will be discussed in greater detail below.

FIG. 4 illustrates a headwall system 156 that includes headwall unit 66 and a patient support apparatus 20. Headwall unit 66 is adapted to wirelessly communicate with patient support apparatus 20, but may, in some embodiments, include a cable port adapted to allow a cable from patient support apparatus 20 to be coupled thereto. One example of such a headwall unit with a cable port is disclosed in more detail in commonly assigned U.S. patent application Ser. No. 16/217,203 filed Dec. 12, 2018, by inventor Alexander Bodurka and entitled SMART HOSPITAL HEADWALL SYSTEM, the complete disclosure of which is incorporated herein by reference. In some embodiments, headwall unit 66 includes one or more cable ports and the functionality of the headwall units described in the aforementioned '203 application.

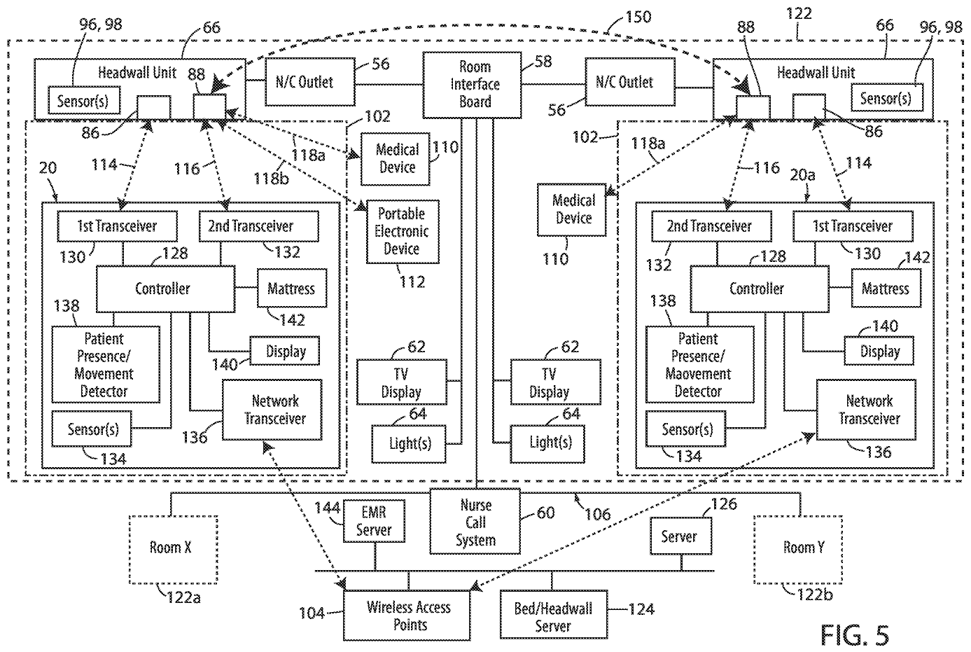

As is also shown in FIG. 4, headwall unit 66 includes a first wireless transceiver 86, a second wireless transceiver 88, a network transceiver 90, a controller 92, a memory 94, an ambient light sensor 96, an ambient sound sensor 98, and a nurse call transceiver 100. First transceiver 86 is adapted to communicate with a patient support apparatus 20 positioned within a bay area 102 (FIG. 5) and second transceiver 88 is adapted to communicate with patient support apparatus 20 and other devices that are positioned in the room in which headwall unit 66 is located (or otherwise positioned within range of second transceiver 88, which may have a range of on the order of one to two dozen feet or so).

Controller 92 is a conventional microcontroller, in at least one embodiment. For example, in one embodiment, controller 92 is any one of the i.MX family of system-on-chip (SoC) processors and/or any one of the Kinetis K60 family of microcontroller units (MCUs), both of which are marketed by Freescale Semiconductor of Austin, Texas. Other microcontroller units, however, may be used. In general, controller 92 includes any and all electrical circuitry and components necessary to carry out the functions and algorithms described herein, as would be known to one of ordinary skill in the art. Such circuitry may include one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions within headwall unit 66, or they may reside in a common location within headwall unit 66. When physically distributed, the components may communicate using any suitable serial or parallel communication protocol, such as, but not limited to, CAN, LIN, Firewire, I-squared-C, RS-232, RS-465, universal serial bus (USB), etc. The instructions followed by controller 92 in carrying out the functions described herein, as well as the data necessary for carrying out these functions, are stored in one or more accessible memories, such as, but not limited to, memory 94.

Memory 94 (FIG. 4), in addition to storing instructions followed by controller 92, stores data received from patient support apparatus 20 and/or from other devices positioned within the room in which headwall unit 66 is positioned. Such data may include patient data, sensor data, device data, alerts, communication preferences, and other data. In some embodiments, memory 94 may also contain a Uniform Resource Locator (URL) that controller 92 transmits to mobile electronic devices, such as a smart phones, carried by caregivers and/or other authorized individuals associated with the healthcare facility. The URL enables the mobile electronic device to easily access and retrieve data stored in memory 94 (or elsewhere) that relates to the patient, patient support apparatus 20, and/or other medical devices associated with headwall unit 66.

Network transceiver 90 (FIG. 4) is adapted to communicate with one or more wireless access points 104 of a healthcare facility network 106. In some embodiments, network transceiver 90 is a WiFi transceiver (IEEE 802.11) adapted to communicate with access points 104 using any of the various WiFi protocols (IEEE 802.11b, 801.11g, 802.11n, 802.11ac . . . , etc.). In still other embodiments, network transceiver 90 is adapted to communicate using any of the frequencies, protocols, and/or standards disclosed in commonly assigned U.S. patent application Ser. No. 62/430,500 filed Dec. 6, 2016, by inventor Michael Hayes and entitled NETWORK COMMUNICATION FOR PATIENT SUPPORT APPARATUSES, the complete disclosure of which is incorporated herein by reference. In still other embodiments, network transceiver 90 may take on other forms and/or protocols.

Nurse call transceiver 100 (FIG. 4) is adapted to communicate with nurse call outlet 56. Nurse call transceiver 100 therefore converts messages from controller 92 into the proper form to match the communication characteristics of nurse call outlet 56. In this regard, nurse call transceiver 100 selects which pin of the multiple pins certain data is to be communicated over and/or converts the data into the proper format and/or protocol for communicating with room interface board 58 and the devices in communication with room interface board 58.

Ambient light sensor 96 (FIG. 4) is a sensor adapted to detect how much ambient light is in the room and/or area of the room in which headwall unit 66 is positioned. Ambient light sensor 96 may be any type of conventional light sensor suitable for the purposes described herein. In some embodiments, ambient light sensor 96 may be adapted to detect how much light is in a particular portion of the room, such as the portion in front of headwall unit 66 where patient support apparatus 20 is typically positioned. Ambient light sensor 96, as will be discussed in greater detail below, is adapted to detect how much light the patient assigned to patient support apparatus 20 is exposed to during that patient's stay at the healthcare facility. To that end, ambient light sensor 96 is adapted to repetitively take light readings throughout the day and night so that a record is generated of the light intensity levels the patient is exposed to throughout the entirety of their stay in that room. This gives the healthcare facility administrators a record of how dark the room was at night, and for how long, as well as how much light was experienced during daytime hours. Such information, particularly the former, is useful for assessing causes of a patient's ability or inability to sleep at night while in the healthcare facility, as well as for determining potential correlations between light levels and the patient's perceived comfort levels while staying at the healthcare facility. This information may also be useful for predicting and/or improving the scores generated from patient questionnaires about their visits to the healthcare facility (for example, Hospital Consumer Assessment of Healthcare Providers Systems (HCAHPS) scores).

As will further be discussed below, the light readings taken by ambient light sensor 96 are initially stored in memory 94 and later transferred to one or more servers on local area network 106, such as, but not limited to, a bed/headwall server 124 and/or another server 126. The particular path by which these light sensors readings are forwarded to the local area network 106 may vary. In some embodiments, headwall unit 66 communicates these readings to patient support apparatus via one or both of transceivers 86, 88, and then patient support apparatus 20 forwards them to network 106 via its own onboard network transceiver. This path may be particularly useful in those situations where headwall unit 66 does not include its own network transceiver 84. In those embodiments where headwall unit 66 includes its own network transceiver 84, the ambient light readings may be forwarded to one or more servers 124, 126 on network 106 via network transceiver 84. In some situations, headwall unit 66 may be adapted to send its readings to another headwall unit 66 via a mesh network using second transceiver 88. Such mesh network routing may be particularly useful when transceiver 84 of headwall unit 66 is not within range of an access point 104 (or headwall unit 66 does not include transceiver 84), when patient support apparatus 20 is not communicatively paired with headwall unit 66, and/or when network congestion makes routing this information to one or more other headwall units 66 more efficient. When mesh network routing between headwall units 66 is utilized, the ambient light sensor readings are eventually transferred from the one of the headwall unit 66 in the mesh network to local area network 106 via either transceiver 84 or via the network transceiver onboard a pair patient support apparatus 20.

Ambient sound sensor 98 (FIG. 4) is a sensor adapted to detect how much ambient sound is in the room and/or area of the room in which headwall unit 66 is positioned. Ambient sound sensor 98 may be any type of conventional sound sensor suitable for the purposes described herein. In some embodiments, ambient sound sensor 98 may be adapted to detect how much sound is in a particular portion of the room, such as the portion in front of headwall unit 66 where patient support apparatus 20 is typically positioned. Ambient sound sensor 98, as will be discussed in greater detail below, is adapted to detect how much sound the patient assigned to patient support apparatus 20 is exposed to during that patient's stay at the healthcare facility. To that end, ambient sound sensor 98 is adapted to repetitively take sound readings throughout the day and night so that a record is generated of the sound intensity levels the patient experiences throughout the entirety of their stay in that room. This gives the healthcare facility administrators a record of how quiet the room was at night, and for how long, as well as how much noise was experienced during daytime hours. Such information, particularly the former, is useful for assessing causes of a patient's ability or inability to sleep at night while in the healthcare facility, as well as for determining potential correlations between sound levels and the patient's perceived comfort levels while staying at the healthcare facility. This information may also be useful for predicting and/or improving the scores generated from patient questionnaires about their visits to the healthcare facility (for example, Hospital Consumer Assessment of Healthcare Providers Systems (HCAHPS) scores).

As with the ambient light readings from sensor 96, the sound readings taken by ambient sound sensor 98 are initially stored in memory 94 and later transferred to one or more servers 124, 126 on local area network 106. The particular path by which these sound sensors readings are forwarded to the local area network 106 may take on any of the same pathways discussed above with respect to the ambient light readings from sensor 96.

It will be understood that, although FIG. 4 illustrates headwall unit 66 with ambient light sensor 96 and ambient sound sensor 98, headwall unit 66 may be modified to include either fewer or greater numbers of sensors. Still further, headwall unit 66 may be modified to include different types of sensors from the two shown in FIG. 4. In some embodiments, headwall unit 66 may include one or more of the following: a temperature sensor, a volatile organic compound (VOC) sensor, a caregiver presence sensor that detects the presence/absence of a caregiver in the room, and/or other types of sensors. In general, headwall unit 66 may include any one or more sensors that provide data indicative of the level of comfort and/or satisfaction that a patient experiences while in the healthcare facility. Still further, it will be understood that, in some embodiments discussed in greater detail below, headwall unit 66 may be modified to omit any such sensors, but instead be utilized in one or more of the manners discussed in further detail below.

Headwall unit 66 is typically positioned at the head of a bay area 102 (FIG. 5), which is the area where the patient support apparatus 20 typically remains when it is positioned within a particular room of the healthcare facility. In some healthcare facilities, one or more of the rooms are single patient support apparatus rooms in which only a single patient support apparatus is present (private rooms). In such rooms, there is only one bay 102. Healthcare facilities, however, typically include one or more rooms in which multiple patient support apparatuses 20 are positioned (semi-private rooms). In such rooms, there are multiple bays 102 for the multiple patient support apparatuses 20.

First wireless transceiver 86 of headwall unit 66 is part of first wall unit 68 and is adapted to communicate with patient support apparatus 20. In the illustrated embodiment, first transceiver 86 is implemented as an infrared transceiver and is adapted to communicate with patient support apparatus 20 only when patient support apparatus 20 is positioned within close proximity to headwall unit 66, such as when patient support apparatus 20 is positioned within the particular bay area 102 associated with that particular headwall unit 66. In other embodiments, it will be understood that first wireless transceiver 86 of headwall unit 66 may be implemented using short range communication media and/or protocols other than infrared communications, including, but not limited to, optical communications.

Each headwall unit 66 includes a unique identifier 108 that uniquely identifies that particular headwall unit 66 from the other headwall units 66 within the healthcare facility. This unique identifier is used by patient support apparatus 20 to communicatively pair with second transceiver 88 and/or to determine its location within the healthcare facility. When first transceiver 86 is able to communicate with patient support apparatus 20, controller 92 of headwall unit 66 transmits the unique identifier 108 to the patient support apparatus 20 (or other device). In the embodiment shown, unique identifier 108 is sent to patient support apparatus 20 only via first transceiver 86. It will be understood, however, that both first and second transceiver 86 and 88 may transmit identifier 108 to the patient support apparatus 20.

In order to determine location from the unique identifier, a controller on board patient support apparatus 20 (discussed more below) sends the unique identifier to one or more servers 124, 126 on a healthcare facility computer network 106 (via an onboard network transceiver), and the server 124, 126 converts the identifier into a location via a look-up table it has access to that correlates all of the headwall unit 66 identifiers within the healthcare facility to their respective locations. Alternatively, the controller on board patient support apparatus 20 consults an on-board look-up table that correlates the unique identifiers to locations within the healthcare facility and the controller determines the location of patient support apparatus 20 via the look-up table. In still another embodiment, unique identifier 108 identifies directly the room number in which headwall unit 66 is positioned, as well as the bay area 102 associated with headwall unit 66, and patient support apparatus 20 does not have to consult a look-up table to convert the ID 108 into a location, but instead is able to determine its location directly from the ID 108.

Second transceiver 88 of headwall unit 66 is adapted to communicate with patient support apparatus 20 using a second communication link 116. Second communication link 116 is a Radio Frequency (RF) communication link that is not line-of-sight, unlike the IR communication link 114 of first transceiver 86. In some embodiments, second transceiver 88 is a Bluetooth transceiver configured to communicate using one or more of the Bluetooth standards (e.g. IEEE 802.14.1 or any of the standards developed by the Bluetooth Special Interest Group). It will be understood, however, that in other embodiments, second wireless transceiver 88 may utilize other forms of Radio Frequency (RF) and non-RF communication. For purposes of the following written description it will be assumed that second transceiver 88 communicates using conventional Bluetooth technology (including, but not limited to, Bluetooth Low Energy), although this written description is not meant to be an indication that other types of communication cannot be used between second transceiver 88 and the off-board devices with which it communicates, such as, but not limited to, patient support apparatus 20, one or more medical devices 110, and/or one or more mobile electronic devices 112 associated with caregivers, patients, visitors, technicians, and/or other people.

In some embodiments, first communication link 114 is used by headwall unit 66 to establish and periodically verify that patient support apparatus 20 (and/or other devices) are within bay area 102, while second communication link 116 is used to communicate information back and forth between headwall unit 66 and patient support apparatus 20 (and other devices within the room). In some of these embodiments, first communication link 114 may alternatively be used in situations where second communication link 116 is blocked or otherwise not functional. In still other modified embodiments, first communication link 114 may be used to communicate data in addition to the location identifier 108, as well as to perform other functions besides establishing and verifying the presence of patient support apparatus 20 and/or other devices within bay area 102. In some embodiments, first transceiver 86 transmits unique identifier 108 and/or other information wirelessly to patient support apparatus 20 and patient support apparatus 20 uses that information to establish communication link 116 (e.g. pair) with second transceiver 88. In other words, in some embodiments, first transceiver 86 is used to communication information to patient support apparatus 20 that permits out of band pairing between patient support apparatus 20 and second transceiver 88 for establishing second communication link 116. Examples of such out-of-band pairing, which may be utilized with headwall unit 66, are disclosed in more detail in commonly assigned U.S. patent application Ser. No. 16/215,911 filed Dec. 11, 2018, by inventors Alexander Bodurka et al. and entitled HOSPITAL HEADWALL COMMUNICATION SYSTEM, the complete disclosure of which is incorporated herein by reference.

In addition to other functions, first and second transceivers 86 and 88 are utilized by controller 92 of headwall unit 66 to communicate information wirelessly to patient support apparatus 20 and to receive information wirelessly from patient support apparatus 20. In some instances, the information received from patient support apparatus 20 is forwarded to room interface board 58 via nurse call transceiver 100, while in other instances, the information received from patient support apparatus 20 is stored in memory 94. In still other instances, the information received from patient support apparatus 20 is both stored in memory 94 and forwarded to room interface board 58, which may in turn forward the information to nurse call system 60 and/or other devices in communication with room interface board 58.

When patient support apparatus 20 is positioned within a bay 102 and in normal communication with headwall unit 66, both of the transceivers 86 and 88 are in communication with patient support apparatus 20, and second transceiver 88 may also be in communication with a medical device 110 and/or a mobile electronic device 112 carried by a person (e.g. a smart phone, tablet computer, laptop, etc.) via an auxiliary communication link 118. If patient support apparatus 20 is positioned outside of the bay area 102, first transceiver 86 will not be able to communicate with patient support apparatus 20 because first transceiver 86 uses infrared signals, which are line-of-sight signals, and first transceiver 86 is set up such that its line-of-sight signals are only detectable by the patient support apparatus 20 when the patient support apparatus 20 is positioned within the corresponding bay 102, or a portion of that bay 102. Accordingly, when controller 92 determines that first transceiver 86 is able to successfully communicate with a patient support apparatus 20, it concludes that the patient support apparatus 20 is positioned adjacent to the headwall unit 66.

Second transceiver 88 is able to communicate with patient support apparatus 20 when patient support apparatus 20 is positioned outside of bay area 102 because second transceiver 88 is a Bluetooth transceiver that uses radio frequency (RF) waves that are not line-of-sight. Accordingly, none of patient support apparatus 20, medical devices 110, and/or mobile electronic device 112 needs to be in bay area 102 to communicate with second transceiver 88. The power levels of the Bluetooth communication used by second transceiver 88 may be sufficient to allow patient support apparatus 20, medical devices 110, and/or mobile electronic device 112 to be able to communicate with second transceiver 88 when these devices are positioned outside of the room in which the headwall unit 66 is positioned, depending upon the size of the room and/or the position of headwall unit 66 within the room.

Headwall unit 66 is adapted, in some embodiments, to be a repository of information relating to the patient associated with patient support apparatus 20, the patient support apparatus 20 itself, and one or more medical devices 110 used in conjunction with the treatment of the patient. As such, headwall unit 66 is configured to establish communications with patient support apparatuses 20 and medical devices 110 when these devices are brought into a room. After being brought into the room, data from these devices is transmitted to headwall unit 66 and, in some embodiments, stored locally in memory 94, and in other embodiments is transmitted to a remote location (either in lieu of, or in addition to, storing the data locally).

Second transceiver 88 is configured to periodically transmit a beacon signal, such as, but not limited to, approximately once every second. The beacon signal includes a beacon ID 120. Beacon ID 120, in some embodiments, is different from the IR ID 108. In other embodiments, beacon ID 120 is the same as IR ID 108. The beacon ID 120 is sent in order to allow headwall unit 66 and one or more devices 110 and/or 112 to establish auxiliary communication links 118 between themselves. In the embodiment illustrated in FIG. 4, second transceiver 88 has established a first auxiliary communication link 118a between itself and a medical device 110, and a second auxiliary communication link 118b between itself and a mobile electronic device 112. In alternative embodiments, second transceiver 88 transmits beacon signals to devices 110 and/or 112 without establishing a communication link with them. In such alternative embodiments, second transceiver 88 merely transmits the beacon ID 120 to the devices 110 and/or 112 and does not establish any two-way communication with the devices.

In those embodiments where an auxiliary communication link 118 is established between second transceiver 88 and one or more devices 110 and/or 112, such links are 118 are automatically established in response to the beacon signal. That is, headwall unit 66 is configured, in at least some embodiments, to automatically establish a communication link 118 between second transceiver 88 and any medical device 110 and/or mobile electronic device 112 that enters the same room as headwall unit 66, or that otherwise comes within range of second transceiver 88. This automatic communication link establishment takes place without requiring any steps on the part of a caregiver that are specific to this process. In other words, the caregiver does not need to press a button, flip a switch, or manipulate any controls on patient support apparatus 20, headwall unit 66, medical device 110, or portable electronic device 112 to establish link(s) 118. Instead, the mere positioning of devices 110, 112 within range of second transceiver 88 automatically causes link 118 to be established.

In those situations where one or more rooms in a healthcare facility contain multiple headwall units 66 (see, e.g., room 122 of FIG. 5), multiple second transceivers 88 may establish auxiliary communication links 118 with the same medical device 110 and/or the same portable electronic device 112. This is also true in those situations where the range of a second transceiver 88 on a first headwall unit 66 overlaps with the range of another second transceiver 88 on a second headwall unit 66, but the first and second headwall units are not positioned in the same room. In either situation, the medical device 110 and/or portable electronic device 112 may be in communication with multiple second transceivers 88 of multiple headwall units 66.

The communication of devices 110 and/or 112 with multiple second transceivers 88 of multiple headwall units 66 stands in contrast with the communication of patient support apparatuses 20 with second transceivers 88. That is, each patient support apparatus 20 will communicate with only a single second transceiver 88 of a single headwall unit 66 after the patient support apparatus 20 is moved to a particular bay area 102, despite the fact that, while positioned within that bay area, the patient support apparatus 20 is within range of multiple second transceivers 88 of multiple headwall units 66. This is because, as the patient support apparatus 20 moved to its intended bay area 102, the patient support apparatus 20 establishes link 114 with first transceiver 86, and first transceiver 86 transmits to the patient support apparatus the unique identifier 108 corresponding to the headwall unit 66 of that particular bay area 102. The patient support apparatus 20 uses this specific identifier to determine which of the multiple headwall units 66 it is supposed to have second communication link 116 with, and disestablishes any second communication links 116 it may have temporarily established with the other headwall unit(s) 66 that do not have the specific identifier it received via communication link 114. The result is that patient support apparatus 20 ends up having a single communication link 116 and a single communication link 114 with only one (and the same) headwall unit 66.

Regardless of the specific links implemented, when patient support apparatus 20 is positioned in front of a headwall unit 66 in bay area 102, it communicates both messages to be relayed to room interface board 58 (which then routes them accordingly) and data to be stored within headwall unit 66. The data to be stored within headwall unit 66 includes data about the status of one or more sensors, switches, actuators, and other components of patient support apparatus 20, as well as data about the patient associated with patient support apparatus 20. The specific data sent by patient support apparatus 20 to be stored by headwall unit 66 may vary widely from embodiment to embodiment. In general, this data may include any one or more of the following items of information: the status of a brake on patient support apparatus 20, the status of an exit detection system of patient support apparatus 20 (e.g. whether armed or not); the status of siderails 34 (e.g. whether raised or not); the height of support deck 30; the angle of head section 42; any of one or more weights detected or calculated by a scale system on board patient support apparatus 20 (e.g. a tare weight, patient weight, object weight, etc.); a mobility score or index of the patient; one or more vital signs of the patient; a time at which the patient was last turned; any data from a powered mattress positioned on board patient support apparatus 20 (e.g. inflation pressure, therapies implemented via the powered mattress, patient movement detected by the mattress, etc.); a time since the patient last exited patient support apparatus 20; an amount of time the patient spent off patient support apparatus 20; one or more measurements related to a patient's sleep (e.g. quantity, quality, start time, end time, etc.); interface pressures between the patient and the mattress, or other surface, on which the patient is supported; a cleaning status of patient support apparatus 20; an identity of the patient (as detected by the patient support apparatus 20 or entered via a caregiver into a memory on board the patient support apparatus); and still other data gathered from one or more components of patient support apparatus 20.

The sensors and components used to generate any of the above-identified information may vary from patient support apparatus to patient support apparatus, and some patient support apparatuses may be able to detect and transmit more information to headwall unit 66 than other patient support apparatuses. Although other constructions may be used, the following chart identifies commonly assigned U.S. patent applications/patents disclosing patient support apparatuses constructed to detect the identified data. Any of these constructions may be used by patient support apparatus 20 to detect the aforementioned data that is forwarded to headwall unit 66, although other constructions may also be used. Each of these commonly assigned patent applications is incorporated herein by reference in their entirety. Still other data may be detected and forwarded to headwall unit 66 beyond the data identified above and the data detected in the following patents and patent applications.

| Patent/App. | Filing Date | Title | Data Detected |
|---|---|---|---|
| 5,276,432 | Jan. 15, 1992 | Patient Exit Detection Mechanism | Patient's location (center of gravity) |
| | | for Hospital Bed | |
| 7,699,784 | Jul. 5, 2007 | System for Detecting and | Patient's heart rate, breathing rate, and |
| | | Monitoring Vital Signs | other vital signs |
| 9,320,444 | Mar. 14, 2014 | Patient Support Apparatus with | Patient sleep quantity, quality, and |
| | | Patient Information Sensors | other sleep parameters; patient weight |
| 61/449,182 | Mar. 4, 2011 | Sensing System for Patient | Patient interface pressures, vital signs, |
| | | Supports | |
| 14/692,871 | Apr. 22, 2015 | Person Support Apparatus with | Patient movement |
| | | Position Monitoring | |
| 14/873,734 | Oct. 2, 2015 | Person Support Apparatus with | Patient and object weights, movement, |
| | | Motion Monitoring | and position |
| 14/928,513 | Oct. 30, 2015 | Person Support Apparatus with | A patient's activity, time out of bed, |
| | | Patient Mobility Monitoring | number of steps, and other activity data |
| 14/578,630 | Dec. 22, 2014 | Video Monitoring System | Patient turns, bed sore assessment |
| | | | scores, eating and sleeping, exit |
| | | | detection system status, etc. |
| 15/346,779 | Nov. 9, 2016 | Person Support Apparatus with | Patient vital signs, position, movement |
| | | Acceleration Detection | |
| 15/809,351 | Nov. 10, 2017 | Patient Support Apparatuses with | Patient mobility score and/or |
| | | Mobility Assessment | assessments |
| 15/709,586 | Sep. 20, 2017 | Systems and Methods for | Cleanliness and/or usability status of a |
| | | Determining the Usability of | patient support apparatus |
| | | Person Support Apparatuses | |

In addition to the aforementioned data, headwall unit 66 may be adapted to receive data from one or more medical devices 110 positioned within the room and associated with the patient on patient support apparatus 20. Such data, as well as the processing of such data, the retention of such data, and the manner in which such data is transmitted to headwall unit 66 and/or elsewhere, may be the same as is disclosed in commonly assigned U.S. patent application Ser. No. 16/217,203 filed Dec. 12, 2018, by inventor Alexander Bodurka and entitled SMART HOSPITAL HEADWALL SYSTEM, the complete disclosure of which is incorporated herein by reference.

In some embodiments, headwall unit 66 is configured to permit communication access to nurse call outlet 56 only to patient support apparatuses 20, and not to medical devices 110 and/or portable electronic devices 112. In other words, when patient support apparatus 20 establishes communication link 116 with a particular headwall unit 66, the patient support apparatus 20 is able to pass data to headwall unit 66 that headwall unit 66 then forwards to nurse call outlet 56. This data may take the form of alerts and/or status information that is passed to nurse call system 60, or it may take the form of commands to television 62, and/or it may take on other forms. In contrast, when a medical device 110 and/or a mobile electronic device 112 establishes a communication link 118 with the headwall unit 66, headwall unit 66 is configured to not forward any communications from those devices 110 and/or 112 to nurse call outlet 56. Thus, headwall unit 66 does not grant access to nurse call outlet 56 (and the structures communicatively coupled thereto, e.g. room interface board 58, nurse call system 60, television 62, etc.) to medical devices 110 and/or portable electronic devices 112.

Headwall unit 66 may be configured to distinguish between communication link 116 with a patient support apparatus 20 and an auxiliary communication link 118 with a medical device 110 or portable electronic device 112 in different manners. In one manner, headwall unit 66 utilizes a unique ID received from a patient support apparatus 20 that distinguishes it from medical devices 110 and/or portable electronic devices 112. In another manner, either in addition to, or in lieu of the unique patient support apparatus ID, headwall unit 66 utilizes the IR ID 108 it receives over first communication link 114 to distinguish between patient support apparatus 20 and devices 110 and/or 112. In such embodiments, each patient support apparatus 20 may be adapted to use the IR ID it receives from headwall unit 66 via link 114 as a cryptographic key, an input to a hash function, or as another input that it uses to generate a different key, number, or other unique sequence of characters. That different key, number, or unique sequence of characters is generated on patient support apparatus 20 in a unique manner using a unique algorithm, and is then forwarded by patient support apparatus 20 to headwall unit 66 using second communication link 116. Headwall unit 66 includes knowledge of the secret algorithm used by the patient support apparatus 20, and it independently uses this secret algorithm to generate its own key, number, or unique sequence of characters. It then can check to see if the key, number, or unique sequence of characters, it received from the patient support apparatus 20 matches what it generated itself. If it does, headwall unit 66 concludes it is talking to an authorized patient support apparatus 20 and grants it permission to forward data to nurse call outlet 56 (and also forwards data received from nurse call outlet 56 to the authorized patient support apparatus 20). If it does not match, headwall unit 66 concludes it is not talking to an authorized patient support apparatus 20 and it does not allow it to forward information to nurse call outlet 56, nor does it share information it receives from nurse call outlet 56 with that device. Still other manners of distinguishing between communication links 116 with patient support apparatuses 20 and communication links 118 with non-patient support apparatus devices may be utilized.

The types of medical devices 110 which headwall unit 66 and/or patient support apparatus 20 communicate with may vary widely. Some examples of these types of medical devices 110 include, but are not limited to, the following: a ventilator, an infusion pump; a pulse oximeter; a vital sign(s) detector; a Deep Vein Thrombosis (DVT) cuff (or other DVT device); a patient identification device (e.g. an RF ID tag, bracelet, or the like); a pulse wave velocity detector; a sleep detector; a thermal control system; and others. One suitable example of a pulse wave velocity detector that may be used with the patient and that forwards its data to headwall unit 66 is disclosed in commonly assigned U.S. patent application Ser. No. 14/884,222 filed Oct. 15, 2015, by inventors Sean Hadley et al. and entitled SYSTEMS AND METHODS FOR DETECTING PULSE WAVE VELOCITY, the complete disclosure of which is incorporated herein by reference. One suitable example of a thermal control system that may be used with the patient and that forwards its data to headwall unit 66 is disclosed in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

FIG. 5 illustrates in greater detail various internal components of patient support apparatus 20, as well as more of the healthcare IT infrastructure 52 that may be present in a particular healthcare facility. FIG. 5 also illustrates an illustrative non-private room 122 of a healthcare facility and the manner in which the headwall units 66 of the room 122 may interact with their associated patient support apparatuses 20 and the healthcare IT infrastructure. Although FIG. 5 illustrates two identical patient support apparatuses 20 and 20a, it will be understood that this is merely for purposes of explanation and that the particular types of patient support apparatuses 20 used with headwall units 66 may vary within a healthcare facility.

Patient support apparatus 20 includes a controller 128 in communication with a first transceiver 130, a second transceiver 132, one or more sensors 134, a network transceiver 136, a patient presence/movement detector 138, a display 140, and, in some cases, a powered mattress 142. Controller 128, like controller 92 of headwall unit 66, includes any and all electrical circuitry and components necessary to carry out the functions and algorithms described herein, as would be known to one of ordinary skill in the art. Generally speaking, controller 128 may include one or more microcontrollers, microprocessors, and/or other programmable electronics that are programmed to carry out the functions described herein. The other electronic components may include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions within patient support apparatus 20, or they may reside in a common location within patient support apparatus 20. When physically distributed, the components may communicate using any suitable serial or parallel communication protocol, such as, but not limited to, CAN, LIN, Firewire, I-squared-C, RS-232, RS-465, universal serial bus (USB), etc. The instructions followed by controller 128 in carrying out the functions described herein, as well as the data necessary for carrying out these functions, are stored in one or more accessible memories (not shown).

First transceiver 130 of patient support apparatus 20 is adapted to communicate with first transceiver 86 of headwall unit 66. Thus, in some embodiments, first transceiver 130 is an infrared transceiver that communicates with headwall unit 66 only when patient support apparatus is within line-of-sight communication range of first transceiver 86 (e.g. within the corresponding bay 102). Second transceiver 132 of patient support apparatus 20 is adapted to communicate with second transceiver 88 of headwall unit 66. Thus, in some embodiments, second transceiver 132 is a Bluetooth transceiver.

Sensor(s) 134 of patient support apparatus 20 (FIG. 5) may take on a variety of different forms. In some embodiments, sensor(s) 134 include any one or more of the following: a brake sensor adapted to detect whether or not a caregiver has applied a brake to patient support apparatus 20; a height sensor adapted to detect the height of support deck 30 (and/or detect whether support deck 30 is at its lowest height or not); siderail sensors adapted to detect whether siderails 34 are in their raised or lowered orientations; an exit detection status sensors adapted to detect whether an exit detection system on board patient support apparatus 20 is armed or not; a microphone adapted to detect the voice of patient positioned on patient support apparatus 20 so that the patient can communicate aurally with a remotely positioned caregiver (via nurse call system 60); and/or another type of sensor.

Network transceiver 136 of patient support apparatus 20 is adapted to communicate with the wireless access points 104 of healthcare computer network 106 and, in some embodiments, is a WiFi transceiver. Alternatively, network transceiver 136 may take on any of the forms disclosed in commonly assigned U.S. patent application Ser. No. 62/430,500 filed Dec. 6, 2016, by inventor Michael Hayes and entitled NETWORK COMMUNICATION FOR PATIENT SUPPORT APPARATUSES, the complete disclosure of which is incorporated herein by reference.

Patient presence/movement detector 138 is adapted to automatically detect whether or not a patient is currently present on patient support apparatus 20, as well as, in some instances, to detect movement and/or the position of the patient when the patient is supported on patient support apparatus 20. The specific components of patient presence detector 138 and/or manner in which it detects a patient's presence/absence/movement/location may vary from embodiment to embodiment. In one embodiment, patient presence detector 138 includes a plurality of force sensors, such as, but not limited to, load cells that detect the weight and/or center of gravity of the patient. Illustrative manners in which such force sensors can be used to detect the presence and absence of a patient, as well as the center of gravity of the patient, are disclosed in the following commonly assigned U.S. patent references: U.S. Pat. No. 5,276,432 issued to Travis and entitled PATIENT EXIT DETECTION MECHANISM FOR HOSPITAL BED; and U.S. patent application Ser. No. 62/253,167 filed Nov. 10, 2015, by inventors Marko Kostic et al. and entitled PERSON SUPPORT APPARATUSES WITH ACCELERATION DETECTION, the complete disclosures of both of which are incorporated herein by reference. Other algorithms for processing the outputs of the force sensors may also be used for detecting a patient's presence and absence.

Patient presence detector 138 may alternatively be implemented using one or more thermal sensors mounted to patient support apparatus 20 that detect the absence/presence of the patient and/or the position of the patient's head on patient support apparatus 20. Further details of such a thermal sensing system are disclosed in commonly assigned U.S. patent application Ser. No. 14/692,871 filed Apr. 22, 2015, by inventors Marko Kostic et al. and entitled PERSON SUPPORT APPARATUS WITH POSITION MONITORING, the complete disclosure of which is incorporated herein by reference.

In still other embodiments, patient presence detector 138 detects the absence/presence/movement/location of a patient using one or more of the methods disclosed in commonly assigned U.S. patent application Ser. No. 14/928,513 filed Oct. 30, 2015, by inventors Richard Derenne et al. and entitled PERSON SUPPORT APPARATUSES WITH PATIENT MOBILITY MONITORING, the complete disclosure of which is also hereby incorporated herein by reference. In yet other embodiments, patient presence detector 138 includes one or more video cameras for detecting the patient's presence, absence, movement, and/or position, such as disclosed in commonly assigned U.S. patent application Ser. No. 14/578,630 filed Dec. 22, 2014, by inventors Richard Derenne et al. and entitled VIDEO MONITORING SYSTEM, the complete disclosure of which is also hereby incorporated herein by reference. In yet another alternative embodiment, the presence, absence, movement and/or position of a patient is detected using a pressure sensing mat. The pressure sensing mat is positioned on top of the mattress or support deck 30, such as is disclosed in commonly assigned U.S. patent application Ser. No. 14/003,157 filed Mar. 2, 2012, by inventors Joshua Mix et al. and entitled SENSING SYSTEM FOR PATIENT SUPPORTS, the complete disclosure of which is also incorporated herein by reference. In still other embodiments, patient presence detector 138 may take on still other forms.

In some embodiments, display 140 is positioned on patient support apparatus on footboard 32 and is part of footboard control panel 50a. In other embodiments, display 140 is positioned elsewhere, such as one siderails 34 as part of caregiver control panels 50b. In still other embodiments, display 140 is positioned elsewhere. In some embodiments, patient support apparatus 20 includes multiple displays 140. In any of these embodiments, display 140 may be a touch screen display, a non-touch screen display, or any other type of display capable of displaying data gathered from patient support apparatus 20 and/or medical devices 110.

Mattress 142 (which may be the same as mattress 40; see FIG. 2) may be a powered mattress that includes one or more inflatable chambers that are inflated under the control of controller 128 of patient support apparatus 20. In some embodiments, mattress 142 is configured to apply one or more therapies to a patient (e.g. a percussion therapy) and/or to assist in automatically turning a patient. Still further, in some embodiments, mattress 142 may include one or more vital sign sensors built into it that detect one or more of the patient's vital signs, as well as one or more pressure sensors that detect fluid pressure inside of the chambers and/or interface pressure between the patient and the mattress. When mattress 142 includes one or more these features, data from the mattress is communicated to controller 128 which forwards some or all of the data to headwall unit 66 for storage in memory 94 (and/or forwards to a remote location). An illustrative mattress 142 suitable for use with patient support apparatus 20 that includes many of these features is disclosed in commonly assigned U.S. patent application Ser. No. 13/836,813 filed Mar. 15, 2013, by inventors Patrick Lafleche et al. and entitled INFLATABLE MATTRESS AND CONTROL METHODS, the complete disclosure of which is incorporated herein by reference. Other types of mattresses 142 may, of course, be used.

It will be understood that patient support apparatuses 20 include more components than those shown in FIG. 5, and that controller 128 may control more than the components shown in FIG. 5. For example, as noted with respect to FIG. 1, patient support apparatus 20 includes a plurality of control panels 50. Those user interfaces may be in direct communication with controller 128 and/or under the control of controller 128, or those control panels 50 may be under the control of a separate controller that is, in turn, in communication with controller 128. Patient support apparatus 20 may also include an exit detection system that is under the control of controller 128, or that includes its own controller that communicates with controller 128. One such suitable exit detection system is disclosed in commonly assigned U.S. Pat. No. 5,276,432 issued to Travis and entitled PATIENT EXIT DETECTION MECHANISM FOR HOSPITAL BED, which is incorporated herein by reference, although other types of exit detection systems may be included with patient support apparatus 20. Still other components may be present on patient support apparatus 20 and under the control of controller 128 or another controller onboard patient support apparatus 20.

Patient support apparatus 20 is depicted as being located in a particular room 122 of a healthcare facility in FIG. 5. The healthcare facility may include additional rooms 122a, 122b, etc. that are similar to room 122. That is, each room may include one or more headwall units 66, and each headwall unit 66 is in communication with a nurse call outlet 56 and the room interface board 58 for that particular room. The room interface boards 58, in turn, are in communication with room lights 64 and TV 62 for that particular room. Still further, each room interface board 58 is coupled to the nurse call system 60. The nurse call system 60, in some embodiments, is in communication with the healthcare facility computer network 106.

Healthcare facility computer network 106 includes a plurality of servers, such as, but not limited to, an EMR server 144 and headwall/bed server 124. One or more additional servers 126 may also be included, such as, but not limited to, an Internet server and/or an Internet gateway that couples network 106 to the Internet, thereby enabling server 124, headwall units 66, patient support apparatuses 20, and/or other applications on network 106 to communicate with computers outside of the healthcare facility, such as, but not limited to, a geographically remote server operated under the control of the manufacturer of patient support apparatuses 20 and/or headwall units 66. Another type of server that may be included with computer network 106 is a location server (not shown) that is adapted to monitor and record the current locations of patient support apparatuses 20, patients, and/or caregivers within the healthcare facility. Such a location server communicates with the patient support apparatuses 20 and/or headwall units 66 via access points 104 and network transceivers 136 and 84.

Network 106 may also include a conventional Admission, Discharge, and Tracking (ADT) server that allows patient support apparatuses 20 and/or headwall units 66 to retrieve information identifying the patient assigned to a particular patient support apparatus 20. Still further, healthcare network 106 may further include one or more conventional work flow servers and/or charting servers that assign, monitor, and/or schedule patient-related tasks to particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from patient support apparatuses 20 and/or headwall units 66.

It will also be understood by those skilled in the art that still more modifications to network 106 may be made beyond those mentioned herein.

In some embodiments, ambient sound sensors 98 are adapted to only record and/or send sound readings that exceed a user-configurable threshold. In this manner, bandwidth usage can be reduced by only sending sound readings to network 106 (e.g. servers 124 and/or 126) that exceed the threshold, thereby avoiding the sending of sound readings below the threshold. The threshold may be may be chosen such that only sounds that are loud enough to disturb a patient are recorded and/or transferred to one or more servers on network 106. Further, the threshold may be varied throughout the day and/or at different times so that lower thresholds are implemented when the patient is expected to be resting (e.g. at night) and higher thresholds are implemented at times when more noise may be acceptable to the patient (e.g. during day time hours).

Similar to ambient sound sensors 98, ambient light sensors 96 may also be adapted to only record and/or send light readings that exceed a user-configurable threshold and/or that fall below a user-configurable threshold. In this manner, bandwidth usage can be reduced by only sending a subset of the light readings to network 106 (e.g. servers 124 and/or 126) rather than all light readings that are taken. The threshold(s) may be may be chosen such that only light levels that are bright enough to disturb a patient and/or only light levels that are dim enough to make it difficult for a patient to see are transmitted to ness to one or more servers on network 106. Further, the threshold(s) may be varied throughout the day and/or at different times so that, for example, only light above a threshold during evening hours is transmitted to the network 106 and only light below a threshold during daytime hours is transmitted to the network 106. Similar techniques for transmitting only a subset of the sensor data from headwall unit 66 may be used for other types of sensors that are integrated into headwall unit 66.

In some embodiments, the user-configurable thresholds are selected via one or more of the control panels 50 on patient support apparatus 20, in which case the patient support apparatus 20 then forwards the selected thresholds to the adjacent headwall unit 66 via one or both of transceivers 86, 88. Additionally, or alternatively, one or more mobile electronic devices 112 may be used to select the thresholds. In this latter situation, a software app on the mobile electronic device 112 communicates the selected thresholds to the headwall unit 66, either directly through communication with second transceiver 88, or indirectly through patient support apparatus 20 and/or through network 106.

Server 124 and/or server 126 are adapted to receive sound and light recordings (and/or other readings from sensors integrated into headwall units 66) and do one or more of the following: store them, compile them, chart them, analyze them, and make them available to one or more other servers and/or software applications that are in communication with server 124 and/or 126. In carrying out one or more of these functions, server 124, 126 may be configured to execute a caregiver assistance software application (or it may be in communication with a server that is configured to execute a caregiver assistance software application) that is in turn in communication with one or more mobile electronic device (e.g. smart phones, table computers, portable computers). In such embodiments, server 124, 126 may report sounds, light level readings, and/or other information gathered from the sensors of headwall unit 66 to users of such mobile electronic devices. The information may be presented in one or more user-configurable manners, such as being organized by room number, by time period, by patient, by sound levels, by light levels, by work shifts, days of the week, by floors of the healthcare facility, by departments of the healthcare facility, etc. In some embodiments, the caregiver assistance application may be the same as, or include any of the same functionality as (partially or wholly), the caregiver assistance application disclosed in commonly assigned PCT patent application serial number PCT/US2020/039587 filed Jun. 25, 2020, and entitled CAREGIVER ASSISTANCE SYSTEM, the complete disclosure of which is incorporated herein by reference. Still other applications may utilize the data gathered by the sensors of headwall units 66.

In some embodiments, headwall units 66 may be configured to transmit the readings from their sensors (96, 98, etc.) to a server located outside of the healthcare facility. In such cases, that server may perform any of the same functions described above with respect to servers 124 and/or 126.

In some embodiments, headwall units 66 and/or servers 124 and/or 126 are configured to use multiple sound recordings from different headwall unit 66 to triangulate the source of any noises that exceed a threshold, or that persist for a longer than acceptable amount of time. In order to triangulate the location of such noises, controller 92 of headwall units 66 time stamp the recordings they take from their respective sensors 96, 98. By then looking at the sound recordings from multiple headwall units 66 that were taken at the same time and analyzing the magnitude of the respective readings of those headwall units 66 that were able to detect the sound, an estimate of the location of that sound can be determined (using the known location of the headwall units 66 in the healthcare facility). This triangulation may be performed at one or more of the controllers 92 of headwall units 66, or it may be performed at one or more of the servers that receive these time-stamped sound recordings (e.g. servers 124 and/or 126).

In some embodiments, servers 124 and/or 126 are also adapted to receive sensor readings from patient presence detectors 138 and keep track of when, how often, and for how long patients are present on their assigned patient support apparatuses 20. In some such embodiments, the server 124 and/or 126 may be configured to correlate the sound recordings and/or light recordings from sensors 96 and/or 96 with the presence or absence of the patient from patient support apparatus 20. Thus, in some embodiments, servers 124 and/or 126 may filter out excessively loud noises and/or undesirable lighting conditions (e.g. light at night) when the patient is not present on patient support apparatus 20, and thus the patient is unlikely to be experiencing such loud sounds or undesirable lighting.

In some embodiments, headwall units 66 are adapted to communicate directly with one or more other headwall units 66 using either or both of network transceivers 84 and/or second transceivers 88. As shown in FIG. 5, second transceivers 88 may communicate directly with each other over a headwall communication link 150. In such embodiments, controllers 92 may transmit data to each other over links 150 that they receive from any of the onboard sensors 96, 98, etc., or they may transmit any of the data discussed herein that is received from patient support apparatus 20, from nurse call system 60, and/or from any of devices 110 and/or 112. In some embodiments, headwall units 66 use second transceivers 88 and/or network transceivers 84 to generate one or more mesh networks with each other that may be used to forward data to servers 124 and/or 126. In some embodiments, these mesh networks of headwall units 66 may incorporate any of the same algorithms, functions, structures, routing techniques, and/or purposes of the mesh networks implemented on the patient support apparatuses disclosed in commonly assigned U.S. patent application Ser. No. 16/569,225 filed Sep. 12, 2019, by inventors Alexander Bodurka et al. and entitled PATIENT SUPPORT APPARATUS COMMUNICATION SYSTEMS, and/or U.S. patent application Ser. No. 14/559,458 filed Dec. 3, 2014, by inventors Michael Hayes et al. and entitled PATIENT SUPPORT APPARATUS COMMUNICATION SYSTEMS, the complete disclosures of both of which are incorporated herein by reference. Still other types of mesh networking may also be utilized.

Such mesh networking may be used to communicate data from one or more headwall units 66 both to and from one or more servers 124 and/or 126. In some embodiments, the headwall unit 66 closest (by communication link) to the server 124, 126 communicates directly with a wireless access point 104 positioned within range of that headwall unit 66, and the wireless access point 104 routes communications over the network 106 to/from the server 124 and/or 126. In other embodiments, the headwall unit 66 closest (by communication link) to the server 124, 126 communicates directly with an adjacent patient support apparatus 20, using first and/or second transceivers 86, 88, and the patient support apparatus 20 then routes to the communicated data through the nearest access point 104 to server 124, 126. Still other communication routes may be used.

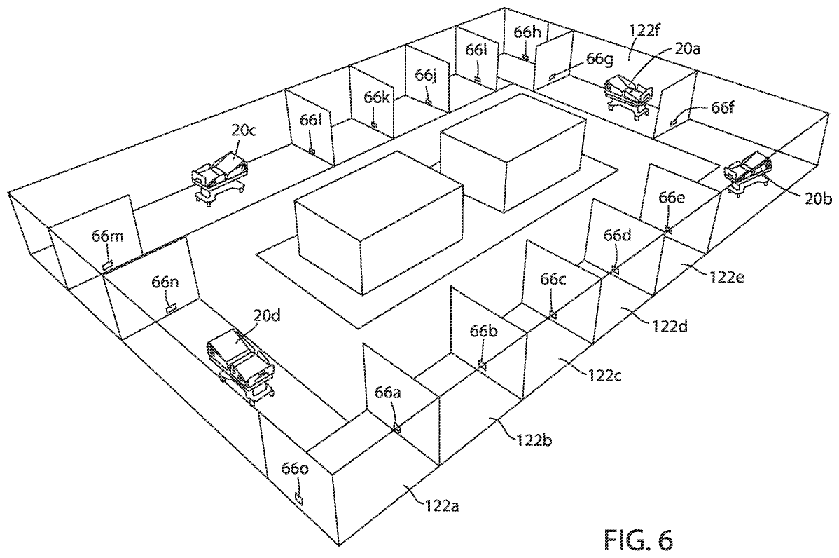

In some embodiments, second transceivers 88 of headwall units 66 emits beacon signals that are utilized by devices other than patient support apparatuses 20 (e.g. devices 110 and/or 112). When multiple headwall units 66 are positioned throughout a healthcare facility, such as is shown in FIG. 6, the headwall units 66 collectively define a network of beacons positioned throughout the healthcare facility. This network of beacons can be utilized by one or more software applications running on one or more smart phones, tablet computers, portable computers, and/or other types of mobile electronic devices 112, and/or by one or more medical devices 110. In some embodiments, headwall units 66 emit the beacon signals from second transceivers 88 using a Bluetooth Low Energy protocol. In such embodiments, second transceivers 88 may utilize a Bluetooth classic protocol for communicating with patient support apparatuses 20, or they may also utilize Bluetooth Low Energy for communicating with patient support apparatuses 20.

In some embodiments, second transceiver 88 uses different Bluetooth profiles or services for communicating different information and/or for communicating with different recipients (e.g. patient support apparatuses 20, medical devices 110, mobile electronic devices 112, etc.). For example, in some embodiments, second transceiver 88 uses a Serial Port Profile (SPP) for communicating data with nurse call outlet 56, a Hands Free Profile (HFP) for communicating audio signals between nurse call system 60 and patient support apparatus 20 of the nurses' and patient's voice, and an Advanced Audio Distribution Profile (A2DP) for communicating audio signals from television 62 to patient support apparatus 20. When communicating with other second transceivers 88 of other headwall units 66, second transceiver 88 may use the Mesh Profile (MESH), and/or other profiles. Still other profiles may be used, including one or more additional profiles for the communication between headwall unit 66 and medical devices 110 and/or mobile electronic devices 112.

In some embodiments, the beacon signals emitted by second transceivers are merely a unique identifier that is translated by the receiving device 110, 112 into a location. In such embodiments, the receiving device 110, 112 contains data, or has access to data, that correlates the unique identifier to a particular location of the healthcare facility. In other embodiments, the beacons emitted by second transceivers transmit additional or different location information, such as a room number, a floor number, a department identifier (e.g. pediatrics, surgery, maternity, intensive care, etc.), a wing identifier, and/or another type of direct location identifier. In these latter embodiments, the receiving device 110, 112 does not need to translate the unique identifier received from headwall unit 66 into a specific location because the beacon signal from headwall unit 66 contains this specific location information already.

Still further, in some embodiments, the beacon signals transmitted from headwall units 66 may enable the devices 110, 112 to establish communications with the headwall units 66 in which headwall units 66 provide additional information to the device 110, 112, such as a floorplan or other type of map of the healthcare facility, including an indication of where the headwall unit 66 is located on the floorplan or other map. Additionally, or alternatively, headwall units 66 may transmit to devices 110, 112 via second transceiver 88 data indicating directions from their current location to other locations within the healthcare facility.

Still further, in some embodiments, headwall units 66 may utilize their direct communication with each other (e.g. via second transceivers 88 and links 150) to provide guidance to a user of a mobile electronic device 112 on how to get to a particular location within the healthcare facility. For example, suppose that a person is located in or adjacent to room 122a in FIG. 6, and that person wishes to get to room 122f. The person may input their desired destination (room 122f) into their mobile electronic device 112 and the device 112 will communicate this information (along with a unique identifier of the device 112) to the headwall unit 66a (and/or 66n) that it is currently in communication with. That headwall unit 66a may then respond with directions to the person to get to room 122f. In addition, headwall unit 66a (or 66n) may then communicate via link 150 with its neighboring headwall unit(s) 66 (e.g. 66b, 66o) that a particular device 112 is seeking to travel to room 122f. This information is communicated along with the unique identifier of the device. The headwall units 66 that receive this information (e.g. 66o, 66b) then forward this information to their neighboring headwall units 66. In the example of FIG. 6, headwall unit 66b would forward this information to headwall unit 66c, and headwall unit 66o would forward this information to headwall unit 66n. These headwall units would then continue to forward this information to all of their neighboring headwall units 66, and so on, until this information was received by the entire network of headwall units 66.

The forwarded information is distributed amongst the headwall units 66 so that the headwall units 66 can provide feedback to the mobile electronic device 112 as it comes within communication range of the various headwall units 66. This feedback may include information that confirms to the user that he or she is on the correct path toward their destination. Likewise, if the user travels in a direction that is not the most direct way to a particular room, the first headwall unit 66 that the mobile electronic device 112 comes into communication range with can send a message to the device 112 indicating that it is traveling in the wrong direction. Thus, for example, if the person starts in room 122a and then moves in a direction where his or her mobile electronic device 112 comes into communication with headwall unit 66n, headwall unit 66n can send a message to the device 112 indicating that it is not taking the most direct route to room 122f. The software onboard device 112 may then query the person if she or she wants to continue in that direction or not. If he or she responds yes, this information may be communicated to headwall unit 66n and headwall unit 66n may communicate with its adjacent headwall units 66 to generate a new route for the person. If he or she responds no, then the device 112 may instruct the user to turn around and travel in the other direction. In such a case, when the user moved with communication range of headwall units 66b, headwall unit 66b would transmit a message to the device 112 indicating that the person was on the most direct path to their destination (room 122f). This type of communication would continue between the device 112 and the headwall units 66 it came into communication range with as the person traveled to their destination.

In alternative embodiments, the software onboard the mobile device 112 might not query the user if he or she wishes to change their path, but instead would simply allow the user to take an alternative path, and headwall units 66 on the alternative path would provide guidance to the destination along the alternative path. Thus, for example, if the user exited room 122a and moved toward headwall unit 66n, headwall unit 66n might send instructions to the device 112 indicating the person is on an alternative route, but that he or she can simply continue to move forward. If he or she did so, headwall units 66m, 66l, 66k, 66j, 66i, and 66h, would eventually provide feedback to the person's device as he or she walked indicating that she was making progress toward his or her destination of room 122f.

In the aforementioned embodiments in which headwall units 66 provide feedback to a person's device 112 about whether or not they are on the correct path to a particular destination, headwall units 66 store location data of all of the other headwall units 66 in their memory 94. This location data indicates not only the room, floor, and/or department of each of the headwall units 66, but also which headwall units 66 are neighbors with each other. From this data, controller 92 is configured to determine the most direct route of a person to a particular destination, including the sequence of headwall units 66 that the person will encounter on his or her travel to the destination. Those headwall units 66 that are on the route will respond with positive feedback to the user's device 112 as that person moves (provided the motion is in the correct sequence), and those headwall units 66 that are not on the route will provide negative feedback to the user and/or result in those headwall units 66 plotting an alternative route for the person.

In some embodiments, not only do headwall units 66 provide textual location information to the mobile electronic devices 112 (e.g. room number, floor number, department, etc.), but they also or alternatively provide graphical location information. Thus, for example, in some embodiments, headwall units 66 contain a graphical map of all or a portion of the healthcare facility in which they are located, and that graphical map includes an indication of where that particular headwall unit 66 is located within the healthcare facility. In such embodiments, the headwall units 66 may be configured to transmit all or a portion of this graphic location data to mobile electronic devices 112 so that the mobile electronic devices 112 can display it on their displays to the user of such devices 112. In this manner, the mobile electronic devices 112 can be programmed to simply display the graphical location data that they receive, rather than programmed with such graphical location data and/or rather than programmed to retrieve such graphical data from another source

It will be understood that the use of the term “transceiver” herein is intended to cover not only devices that include a transmitter and receiver contained within a single unit, but also devices having a transmitter separate from a receiver, and/or any other devices that are capable of both transmitting and receiving signals or messages.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

In the claims:

1. A stationary communication unit adapted to be mounted in a room of a healthcare facility, the stationary unit comprising:
a first transceiver adapted to wirelessly communicate over a first communication channel with a patient support apparatus positioned in the room;
a second transceiver adapted to wirelessly communicate over a second communication channel with the patient support apparatus, the second communication channel different from the first communication channel, the second transceiver further adapted to transmit a message to the patient support apparatus over the second communication channel that includes a first identifier associated with the stationary communication unit;
a third transceiver in communication with a nurse call system outlet installed on a wall of the room, the nurse call system outlet communicatively coupled to a nurse call system, wherein the third transceiver is adapted to receive audio signals from a wire of the nurse call system outlet; and
a controller adapted to forward the audio signals received from the third transceiver to the patient support apparatus over the first communication channel, the controller further adapted to periodically emit a beacon signal from the first transceiver, the beacon signal including a second identifier adapted to be detected by an electronic device other than the patient support apparatus positioned in the room.

2-6. (canceled)

7. The stationary communication unit of claim 1 further including a sensor in communication with the controller, the sensor adapted to detect a parameter relating to the room, and wherein the controller is adapted to forward readings from the sensor to an off-board device using at least one of the first transceiver or the second transceiver.

8. The stationary communication unit of claim 7 wherein the sensor is a sound sensor adapted to detect sound in the room and the off-board device is the patient support apparatus.

9. The stationary communication unit of claim 7 wherein the sensor is a light sensor adapted to detect an amount of light in the room and the off-board device is the patient support apparatus.

10. The stationary communication unit of claim 1 wherein the controller is further adapted to include the first identifier in the audio signals forwarded to the patient support apparatus via the first transceiver, and wherein the electronic devices include a smart phone.

11. (canceled)

12. The stationary communication unit of claim 7 wherein the off-board device is a second stationary communication unit different from the stationary communication unit.

13. A stationary communication unit adapted to be mounted in a room of a healthcare facility, the stationary unit comprising:
a first transceiver adapted to transmit signals to a patient support apparatus;
a second transceiver adapted to transmit a message to the patient support apparatus positioned in the room, the message including a first identifier associated with the stationary communication unit;
a third transceiver in communication with a nurse call system outlet installed on a wall of the room, the nurse call system outlet communicatively coupled to a nurse call system, wherein the third transceiver is adapted to receive audio signals from a wire of the nurse call system outlet;
a sensor adapted to detect a parameter relating to the room; and
a controller adapted to forward the audio signals received from the third transceiver to the first transceiver for transmission to the patient support apparatus, the controller further adapted to forward readings from the sensor to an off-board device.

14. The stationary communication unit of claim 13 wherein the first transceiver is a Bluetooth transceiver, the second transceiver is an infrared transceiver, and the controller is adapted to forward the readings from the sensor to the off-board device using the Bluetooth transceiver.

15. The stationary communication unit of claim 14 wherein the off-board device is one of the patient support apparatus or a second stationary communication unit different from the stationary communication unit.

16. The stationary communication unit of claim 14 wherein the sensor is a sound sensor adapted to detect sound in the room and the off-board device is the patient support apparatus.

17. The stationary communication unit of claim 14 wherein the sensor is a light sensor adapted to detect an amount of light in the room and the off-board device is the patient support apparatus.

18. The stationary communication unit of claim 14 further comprising a WiFi transceiver adapted to communicate with a wireless access point of a local area network, and wherein the off-board device is a server communicatively coupled to the local area network.

19. The stationary communication unit of claim 14 wherein the first transceiver is adapted to transmit signals using a first protocol and a second protocol different from the first protocol, and the controller is adapted to forward the audio signals received from the third transceiver to the patient support apparatus using the first transceiver and the first protocol, and the controller is further adapted to periodically emit a beacon signal from the first transceiver using the second protocol, the beacon signal including a second identifier adapted to be detected by electronic devices positioned in the room.

20-22. (canceled)

23. A stationary communication unit adapted to be mounted in a room of a healthcare facility, the stationary unit comprising:
a first transceiver adapted to wirelessly communicate over a first communication channel with a patient support apparatus positioned in the room;
a second transceiver adapted to wirelessly communicate over a second communication channel with the patient support apparatus, the second communication channel different from the first communication channel, the second transceiver further adapted to transmit a message to the patient support apparatus over the second communication channel that includes a first identifier associated with the stationary communication unit;
a third transceiver in communication with a nurse call system outlet installed on a wall of the room, the nurse call system outlet communicatively coupled to a nurse call system, wherein the third transceiver is adapted to receive audio signals from a wire of the nurse call system outlet; and
a controller adapted to forward the audio signals received from the third transceiver to the patient support apparatus over the first communication channel, the controller further adapted to transmit data to a second stationary communication unit using the first transceiver, the second stationary communication unit different from the stationary communication unit.

24. The stationary communication unit of claim 23 further including a sensor in communication with the controller, the sensor adapted to detect a parameter relating to the room, and wherein the data transmitted to the second stationary communication unit includes data from the sensor.

25. The stationary communication unit of claim 24 wherein the sensor is a sound sensor adapted to detect sound in the room, the controller is further adapted to only transmit the data from the sensor to the stationary communication unit if the sound sensor detects a sound above a threshold, and wherein the stationary communication unit further comprises a clock and the controller is further adapted to transmit a time signal from the clock along with the data from the sensor that is transmitted to the stationary communication unit.

26-27. (canceled)

28. The stationary communication unit of claim 24 wherein the sensor is a light sensor adapted to detect an amount of light in the room.

29. The stationary communication unit of claim 24 wherein the controller is further adapted to include a second identifier with the data transmitted to the second stationary communication unit, the second identifier indicating the room in which the stationary communication unit is located.

30-32. (canceled)

33. The stationary communication unit of claim 29 wherein the first transceiver is a Bluetooth transceiver and the second transceiver is an infrared transceiver.

34. The stationary communication unit of claim 23 wherein the first transceiver is adapted to receive audio signals from the patient support apparatus and forward the received audio signals to the nurse call system outlet using the third transceiver.
